(19) United States
(12) Patent Application Publication
(10) Pub. No.: US 2021/0213163 A1
(43) Pub. Date: Jul. 15, 2021

(54) PRODUCTION METHOD FOR BONE-REGENERATION MATERIAL IMPARTED WITH ANTIMICROBIAL PROPERTIES USING INOSITOL PHOSPHATE, AND ANTIMICROBIAL BONE-REGENERATION MATERIAL PRODUCED BY SAID PRODUCTION METHOD

(71) Applicants: Meiji University, Tokyo (JP); Nagoya Institute of Technology, Nagoya-shi, Aichi (JP); Keio University, Tokyo (JP); ORTHOREBIRTH CO. LTD., Yokohama-shi, Kanagawa (JP)

(72) Inventors: Mamoru AIZAWA, Tokyo (JP); Michiyo HONDA, Tokyo (JP); Tomohiro YOKOTA, Tokyo (JP); Kodai ABE, Tokyo (JP); Mayu UEDA, Tokyo (JP); Toshihiro KASUGA, Nagoya-shi, Aichi (JP); Ken ISHII, Tokyo (JP); Morio MATSUMOTO, Tokyo (JP); Masashi MAKITA, Yokohama-shi (JP)

(73) Assignees: Meiji University, Tokyo (JP); Nagoya Institute of Technology, Nagoya-shi, Aichi (JP); Keio University, Tokyo (JP); ORTHOREBIRTH CO. LTD., Yokohama-shi, Kanagawa (JP)

(21) Appl. No.: 17/055,578

(22) PCT Filed: May 15, 2019
(86) PCT No.: PCT/JP2019/019238

§ 371 Date: Nov. 14, 2020

Related U.S. Application Data
(60) Provisional application No. 62/672,618, filed on May 17, 2018.

(51) Int. Cl. A61L 27/32; A61L 27/02; A61L 27/18; A61L 27/54; A61L 27/58

(52) U.S. Cl. CPC A61L 27/32; A61L 27/02; A61L 27/58; A61L 27/54; A61L 27/18

(57) ABSTRACT

Provided is a bone-regeneration material comprising biodegradable fibers and exhibiting antimicrobial properties at an early stage following surgery, A method for producing a bone-regeneration material having antimicrobial properties and comprising biodegradable fibers, wherein the bone-regeneration material is produced by a step in which the biodegradable fibers are immersed in an inositol phosphate solution, then subsequently immersed in a solution containing silver ions, the biodegradable fibers have an outer diameter of 10-100 μm, contain at least 30 wt % or more of a biodegradable resin and 40 wt % or more of calcium compound particles, and some of the calcium compound particles are exposed on the surface of the biodegradable fibers.

### FIELD OF TECHNOLOGY

Present invention relates to a bone regeneration material having antimicrobial property and a method for producing the antimicrobial bone regeneration material property. The antimicrobial property is imparted by using inositol phosphate.

### BACKGROUND TECHNOLOGY

Conventionally, artificial bone made of calcium compounds such as hydroxyapatite (hereinafter abbreviated as HAp), tricalcium phosphate (hereinafter abbreviated as TCP) has been used as a material for bone regeneration. Recently, a type of material that uses the self-regeneration repair function of the human body by filling a scaffold containing a bone formation promoting factor in a defect portion has been actively used (Patent Document 1).

Since implantation of materials into a human body requires a surgery, there is a risk of bacterial infection after surgery. As a measure to solve this problem, it is possible to impart antimicrobial property to the material to be implanted or to administer antibiotics. Metals such as silver, zinc and copper are used as means for imparting antibacterial properties. In particular, silver has excellent bactericidal activity in ionized form, does not produce resistant bacteria such as methicillin-resistant Staphylococcus aureus (MRSA), and is widely and suitably used because silver has a broad antimicrobial spectrum.

As a method of providing silver on the bone regeneration material, if the material is a metal material such as titanium, the surface coating can be performed by spraying silver metal by using a flame spraying method. However, when the bone regeneration material is composed of biodegradable fibers, it is difficult to use this method because of the effect of high temperature and difficulty of coating to complex shapes.

Recently, there has been developed an implant material for bone regeneration of a type in which biodegradable fibers contain particles of a calcium phosphate compound, and after the material is implanted in vivo, calcium phosphate is eluted together with decomposition and absorption of the biodegradable fibers to promote bone formation. As a method of imparting antimicrobial properties to this type of bone regeneration material, there has been proposed a design in which silver is carried on calcium phosphate particles contained in biodegradable fibers, and the biodegradable fibers are degraded in the body to dissolve the calcium phosphate particles and the silver contained therein is eluted to exert antimicrobial properties (Non-Patent Document 1). However, silver ions should be eluted early after implantation of the bone regeneration material, since the risk of bacterial infection is most serious early after surgery. In the method of Non-Patent Document 1, since silver is contained inside the fiber, there is a possibility that silver ions in an amount necessary for antimicrobial are not eluted early after the operation.

On the other hand, eluted silver ions have antimicrobial properties that kill bacteria, but at the same time they may develop cytotoxicity against osteoblasts and surrounding cells that need to proliferate. Since both antimicrobial and cytotoxicity are more effective as the silver ion concentration increases, it is important to simultaneously satisfy these two mutually conflicting requirements when providing an antimicrobial property to artificial bone.

### PRIOR ART DOCUMENTS

### Patent Document

[Patent Document 1] U.S. Pat. No. 6,162,916

### Non-Patent Literature

[Non-Patent Document 1] Flexible, silver containing nanocomposites for the repair of bone defects: antimicrobial effect against E. coli infection and comparison to Tetracycline, Journal of Materials Chemistry 2008 Oliver D. Schneider et al. etc.

### SUMMARY OF THE INVENTION

### Problem to be Solved by the Invention

Under the circumstances as described above, there has been a need for a highly safe bone regeneration material which exhibits antimicrobial properties early after surgery by carrying silver on a bone regeneration material containing biodegradable fibers and eluting silver ions after the bone regeneration material has been implanted in vivo, and which is less likely to develop cytotoxicity, and a method of manufacturing the same.

### Means for Solving the Problems

In order to solve the above-mentioned problems, the inventors of the present invention have intensively studied, and as a result, have noticed that a portion of the particles of the calcium compound embedded in the fiber is exposed on the surface of the biodegradable fiber containing a considerable amount of the particles of the calcium compound. It has been envisioned that controlled antimicrobial properties can be imparted to the exposed calcium compound particles in the intended amounts by chelating calcium ions (Ca²⁺) and silver ions (Ag⁺) of the calcium compound via hydroxyl groups (OH⁻) of inositol phosphate.

Based on the above idea, inventors of the present invention reached a method for producing a bone regenerating material having antimicrobial properties comprising biodegradable fibers,
the biodegradable fiber having an outer diameter of 10 to 100 μm, containing at least 30% by weight of biodegradable resin and at least 40% by weight of calcium compound particles, and a portion of the calcium compound particles exposed on the surface is immersed in an inositol phosphate solution,
and then immersing the material in a solution containing silver ions.

Further, inventors of the present invention reached materials for bone regeneration having antimicrobial properties including biodegradable fibers.
the biodegradable fiber has an outer diameter of 10 to 100 μm, contains at least 30% by weight of biodegradable resin and at least 40% by weight of calcium compound particles, and a portion of the calcium compound particles is exposed on the surface of the biodegradable fiber, the calcium ions and silver ions of the calcium compound particles exposed on the surface are bound via inositol phosphate, whereby silver is substantially uniformly distributed and fixed on the surface of the biodegradable fiber.

Preferably, the inositol phosphate used herein is IP6.

Preferably, the biodegradable resins used herein are poly-L lactic acid (PLLA) or lactic acid-glycolic acid copolymer (PLGA).

Preferably, the calcium compound used in the present invention is β-phase tricalcium phosphate or calcium carbonate.

Preferably, the bone regenerating material used in the present invention is formed in a cotton wool like structure.

### Advantage of the Invention

Since the bone regeneration material to which the antimicrobial property of the present invention is imparted carries silver on the surface of the biodegradable fiber, the bone regeneration material exhibits effective antimicrobial properties against bacterial infection in an early stage after surgery.

In the bone regeneration material imparted with the antimicrobial property of the present invention, it is possible to appropriately control the balance between the antimicrobial property and the cytotoxicity by adjusting the amount of silver fixed to the surface of the biodegradable fiber by adjusting the concentration of the silver ion solution and the amount of the calcium compound particles contained in the biodegradable fiber.

Since silver is carried only on the surface of the biodegradable fiber in the bone regeneration material imparted with the antimicrobial property of the present invention, elution of silver ions is limited to an early stage after the operation. Therefore, there is little concern that the silver ions will be eluted for a long period of time after the surgery, thereby causing cytotoxicity.

The bone regeneration material having cotton wool like structure that is imparted with the antimicrobial property contains a large amount of calcium compound particles serving as an osteogenic factor, and has controlled antimicrobial/cytotoxicity, and thus is an excellent implant material having high osteogenic ability and high safety.

### BRIEF DESCRIPTION OF THE DRAWINGS

FIG. 1 Appearance photograph of the silver-bearing bone regenerating material of the present invention

FIG. 2 Conceptual diagram of biodegradable fibers constituting the silver-bearing bone regenerating material of the present invention

FIG. 3 A method for producing a material for regenerating silver-bearing bone according to the present invention

FIG. 4 Scanning Electron Microscopy (SEM) Observation Results of the Silver-Supported Bone Regeneration Material of the present invention

FIG. 5 Elemental mapping by energy-dispersive X-ray spectroscopy (EDX) of silver-bearing bone regeneration materials of the present invention

FIG. 6 Elemental analysis by energy dispersive X-ray spectroscopy (EDX) of silver-bearing bone regeneration materials of the present invention

FIG. 7 Results of IP6 adsorption experiments on biodegradable fibers of the bone-regenerating materials of the present invention.

FIG. 8 Method of immobilizing silver ions on bone regenerating material of the present invention

FIG. 9 Results of immobilization experiments of silver ions from the silver-bearing bone reclaimed material of the present invention

FIG. 10 Results of elution experiments of silver ions from the silver-bearing bone regeneration material of the present invention

FIG. 11 Changes over time in the amount of silver ions eluted from the silver-bearing bone regeneration material of the present invention

FIG. 12 Method for evaluating the relative antimicrobial rate of silver-bearing bone reclaimed material of the present invention by using shake method

FIG. 13 Anti-viral evaluation of silver-carrying bone regenerating materials of the present invention by the stop circle method

FIG. 14 Results of antimicrobial evaluation by the inhibition circle method of the silver-bearing bone reclaimed material of the present invention

FIG. 15 Experimental methods for cytotoxicity tests

FIG. 16 Results of cytotoxicity tests with the MTT assay

FIG. 17 Results of cytotoxicity studies

FIG. 18 Method of conducting animal experiment

FIG. 19 Photograph of a tibia removed from a rabbit after the implantation period

FIG. 20 Histological evaluation of animal experiments in which the silver-bearing bone regeneration material of the present invention was implanted in rabbits

FIG. 21 Histological evaluation of animal experiments in which the silver-bearing bone regeneration material of the present invention was implanted in rabbits

FIG. 22 Histological evaluation of animal experiments in which the silver-bearing bone regeneration material of the present invention was implanted in rabbits

FIG. 23 Histological evaluation of animal experiments in which the silver-bearing bone regeneration material of the present invention was implanted in rabbits

FIG. 24 Histological evaluation of animal experiments in which the silver-bearing bone regeneration material of the present invention was implanted in rabbits

FIG. 25 Histological evaluation of animal experiments in which the silver-bearing bone regeneration material of the present invention was implanted in rabbits

FIG. 26 μCT images of animal experiments in which the silver-bearing bone regeneration material of the present invention was implanted in rabbits

FIG. 27 μCT images of animal experiments in which the silver-bearing bone regeneration material of the present invention was implanted in rabbits

FIG. 28 μCT images of animal experiments in which the silver-bearing bone regeneration material of the present invention was implanted in rabbits

FIG. 29 Results of an animal experiment in which a material for silver-bearing bone regeneration of the present invention was implanted into a mouse are shown.

FIG. 30 Results of an animal experiment in which a material for silver-bearing bone regeneration of the present invention was implanted into a mouse are shown.

### DETAILED DESCRIPTION OF THE INVENTION

Hereinafter, embodiments of the present invention will be described with reference to the drawings.

### <Biodegradable Fiber>

The biodegradable fibers of silver-bearing bone regeneration materials of the present invention are produced by spinning polylactic acid or lactic acid-glycolic acid copolymers, such as poly L lactic acid (PLLA) or lactic acid-glycolic acid copolymer (PLGA) as suitable matrix resins using an electrospinning process. In order to allow the calcium compound particles to be contained in a partially exposed state, outer diameter of the fiber is preferably 10 to 100 μm, more preferably 20 to 50 μm. By spinning in a state in which the calcium compound particles are uniformly dispersed and contained in the spinning solution used in the electrospinning method, the particles of the calcium compound can be uniformly dispersed in the biodegradable fiber. When amount of particles of the calcium compound contained in the biodegradable fiber is 40 to 70% by weight, particles of the calcium compound are exposed on the surface of the fiber.

### <Material for Bone Regeneration>

As the material of silver-bearing bone regenerating material of the present invention, biodegradable fibers spun by an electrospinning method using a spinning solution containing calcium compound particles can be suitably used. Electrospun biodegradable fibers are deposited in a cotton wool like structure on a collector of an electrospinning device and collected to form a cotton wool-like bone regenerating material. The material is manufactured and sold under ReBOSSIS trademark by one of the applicants of the present application, for example, and is widely used in actual clinical practice as a bone defect filler material excellent in handleability for an operator.

### <Inositol Phosphate>

The inositol phosphoric acid used to support silver on the bone regeneration material of the present invention refers to inositol phosphorylated with a hydroxyl group, and includes inositol trisphosphate (IP3 C₆H₁₅O₁₅P₃), inositol pentachyphosphoric acid (IP5 C₆H₁₇O₂₁P₅), and phytic acid (IP6 c₆H₁₈O₂₄P₆). Phytic acid (IP6) is inexpensive and has the largest number of hydroxyl groups to be chelated, so that it can be used particularly suitably.

### <Calcium Compound>

As the calcium compound used in the bone regeneration material of the present invention, calcium phosphate, calcium carbonate, and silicon eluting calcium carbonate are suitably used. β-phase tricalcium phosphate (β-TCP) is particularly preferred in terms of osteogenic potential. It is preferable that the size of the calcium compound particles is 1 to 4 μm in order to contain the calcium compound particles in a biodegradable fiber in a state in which a part of the particles is exposed on the surface of the fiber.

### Embodiments of Present Invention

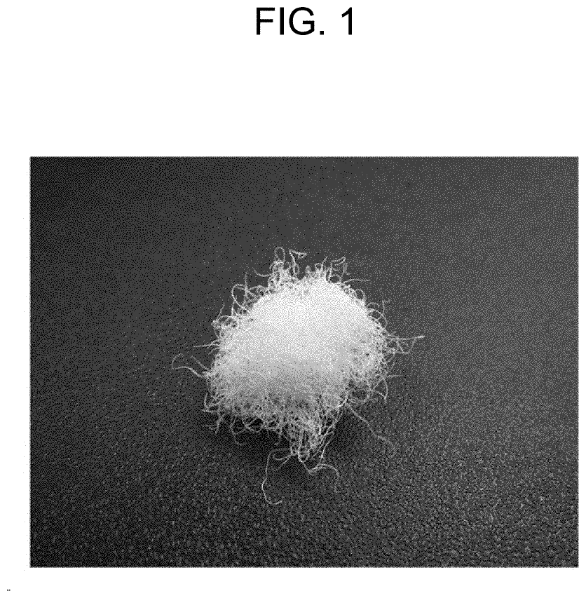

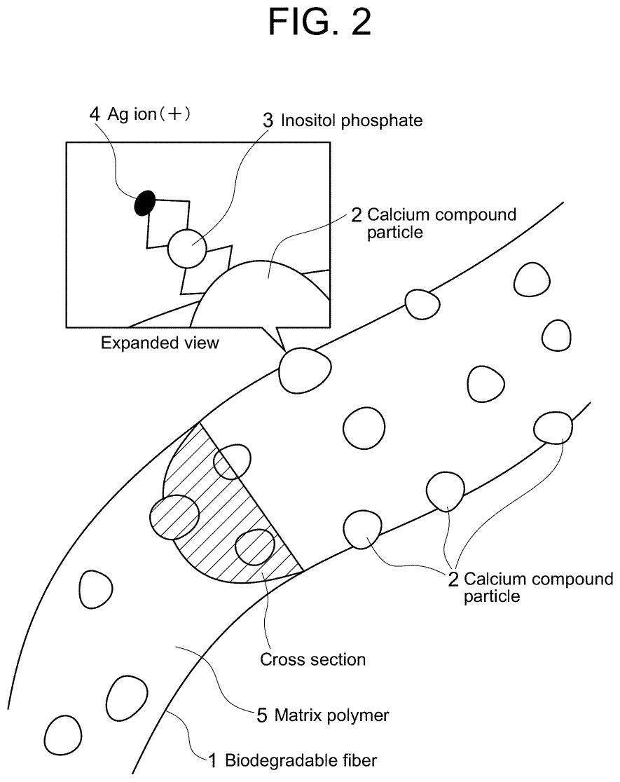

FIG. 1 is a photograph showing the appearance of a material for regenerating bone in a cotton wool like state, which is a preferred embodiment of the present invention. FIG. 2 is a conceptual diagram of biodegradable fibers constituting a silver-bearing bone regeneration material according to an embodiment of the present invention.

Referring to FIG. 2, a biodegradable fiber 1 of silver-bearing bone regeneration material of the present invention includes calcium compound particles 2 in a matrix resin 5, and calcium compound particles 2 are partially exposed on the surface of the biodegradable fiber 1. The calcium ion (Ca²⁺) and the silver ion 4 (Ag⁺) of the calcium compound particles 2 are cross-linked by a chelating bond with a hydroxyl group (OH⁻) of inositol phosphate 3.

When the bone regenerating material of the present invention is implanted into a body, the biodegradable fiber 1 is dissolved over time, resulting in release of inositol phosphate 3. When silver ion 4 is dissolved in a state that it is chelate bonded with inositol phosphate 3 in the presence of calcium ions, bonding between inositol phosphate and silver is cut and silver ion 4 is replaced with calcium ion, because chelate of inositol phosphate 3 is more stable (chelating stability is higher) when it is bonded with Ca²⁺ than when it is bonded with Ag⁺. As a result, silver ion is eluted and exerts an antimicrobial effect.

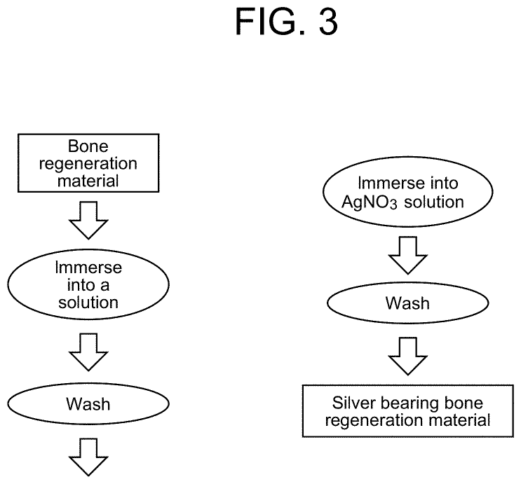

FIG. 3 illustrates a method of producing a silver-bearing bone regeneration material according to an embodiment of the present invention. A predetermined amount of weighed fluffy bone-regenerating material is transferred to 6 well plates, to which 1000 ppm of an aqueous solution of phytic acid (pH 7; 6 ml) was added, and incubated at 37° C. for 24 hours. Thereafter, the solution was washed five times with the same volume of pure water, then immersed in an aqueous silver nitrate solution (0 to 20 mM; 6 mL) at a predetermined concentration, washed five times with the same volume of pure water, and air-dried.

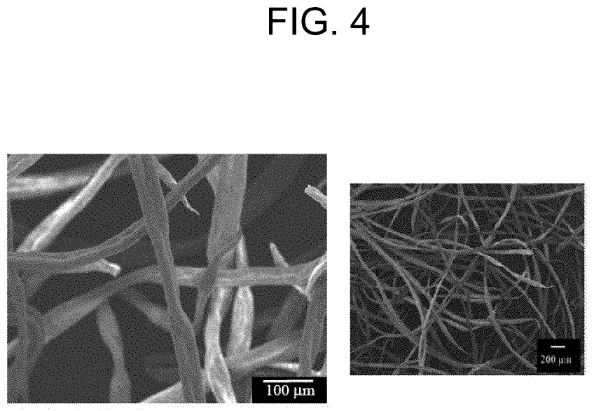

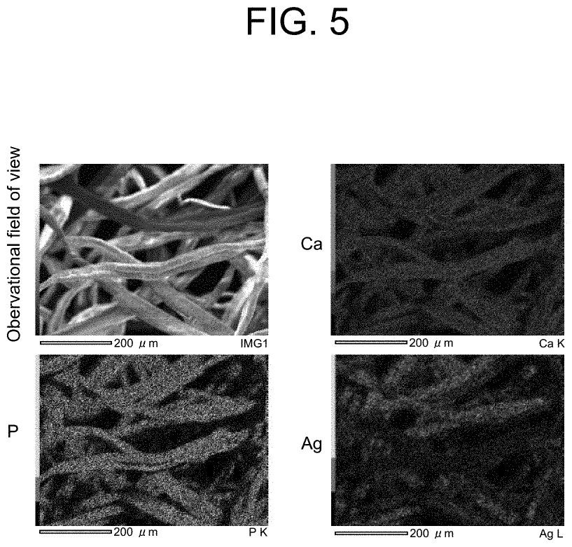

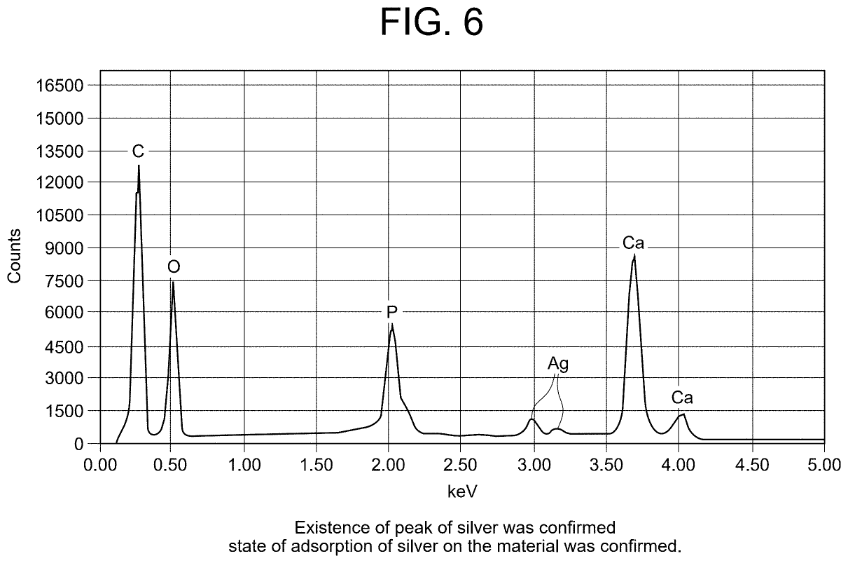

FIG. 4 shows a scanning electron micrograph of the microstructure of the silver-bearing bone regeneration material produced according to the procedure described above. FIG. 5 shows elemental mapping by energy dispersive X-ray spectroscopy (EDX) of the silver-bearing bone regeneration material. FIG. 6 shows the result of confirming the presence of silver ions in the silver-bearing bone regeneration material by elemental analysis of EDX. From the results of the microstructure, elemental mapping, and elemental analysis of the silver-bearing bone regeneration material shown in these figures, it can be seen that the surface structure of the bone regeneration material hardly changes even after the silver ions are immobilized. In addition, it is clear from the EDX analysis of the distribution of elements in the field of view that calcium, phosphorus, and silver are distributed in a distribution corresponding to the fiber shape.

When a biodegradable fiber is immersed in an aqueous silver nitrate solution, a negatively charged functional group (carboxyl group, carbonyl group, or the like) of the biodegradable resin is bonded to Ag⁺ and silver is attached to the surface of the fiber, but since the ionic bond between the silver ion and the functional group of the biodegradable resin is weak, the silver attached to the surface of the resin is not fixed, and is detached by cleaning with pure water. As a result, it is considered that only silver immobilized by chelating with the calcium compound particles via phytic acid remains on the surface of the biodegradable fiber after washing.

### Experiment

### <Adsorption of Inositol Phosphate to Biodegradable Fibers>

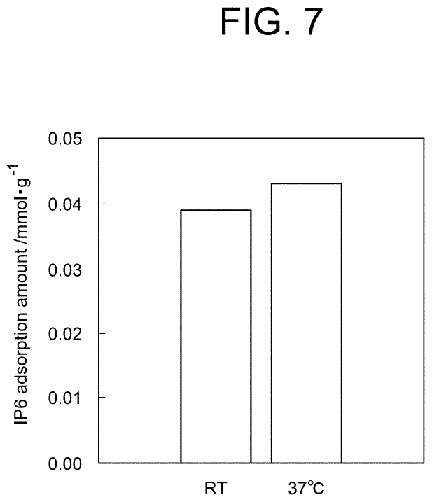

6 well plate was loaded with 0.15 g samples of fluffy bone-regenerating materials (ReBOSSIS® PLLA 30 wt %/βTCP40 wt %/silicon eluting calcium carbonate 30 wt %) and immersed in 1000 ppm concentration 6 ml IP6 solutions and left for 24 hours at room temperature (or 37° C.) humidified condition. Thereafter, IP6 solution not adsorbed to the fiber was recovered and removed. Phosphate ion concentrations of IP6 solution prior to immersion and the recovered solution were measured by inductively coupled plasma-emission spectroscopy. FIG. 7 shows the results of calculating IP6 adsorbed amounts by the phosphate ion concentrations before and after immersion. As shown in FIG. 7, at 25° C. and room temperature, adsorption of slightly less than 0.04 mmol/g of IP6 was observed. In contrast, at 37° C. humidified conditions, IP6 adsorbed slightly more than 0.04 mmol/g.

### <Immobilization of Silver on Biodegradable Fibers by Inositol Phosphate>

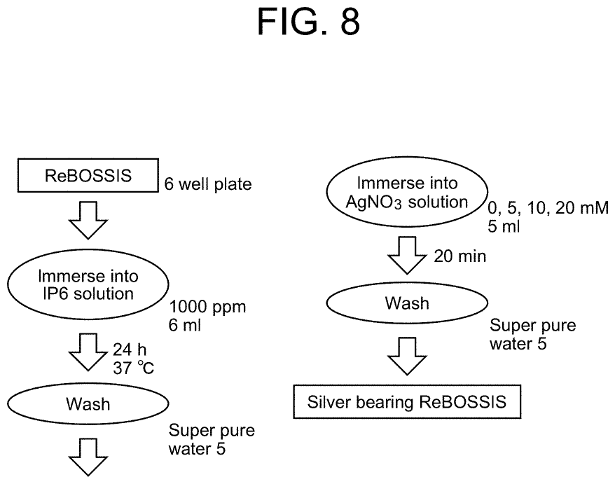

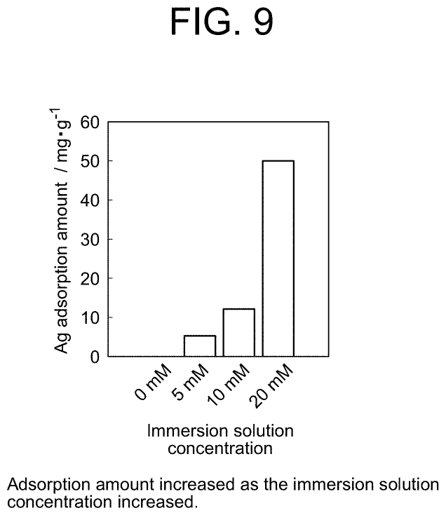

According to the procedures shown in FIG. 8, samples of IP6 adsorbed fluffy bone-regenerating materials (ReBOSSIS®) were placed on a 6 well plate, immersed in 5 ml of an aqueous silver nitrate solution (concentrations: 0, 5, 10, 20 mM), and allowed to stand for 20 minutes. Thereafter, an unabsorbed silver nitrate aqueous solution was recovered and removed from the fiber, and the amount of silver adsorbed was measured by ICP. As shown in FIG. 9, the adsorption amount increased as the concentration of the immersed silver nitrate aqueous solution increased.

### <Elution of Silver Immobilized by Inositol Phosphate>

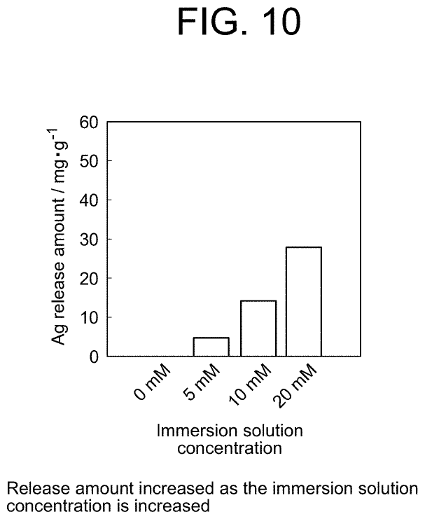

According to the procedures shown in FIG. 8, samples of bone-regenerating materials (ReBOSSIS) loaded with varying amounts of silver (silver nitrate aqueous solution concentrations: 0, 5, 10, 20 mM) using IP6 were immersed in 20 mM HEPES buffer adjusted to pH 7.3 at 37° C. for 24 h (0.01 g/ml), and the eluted amounts of silver ions under neutral conditions were investigated by ICP-AES. The results are shown in FIG. 10. The higher the concentration of silver nitrate, the higher the elution amount of silver ions. The samples treated with 0, 5 and 10 mM had nearly identical amounts of adsorption and elution.

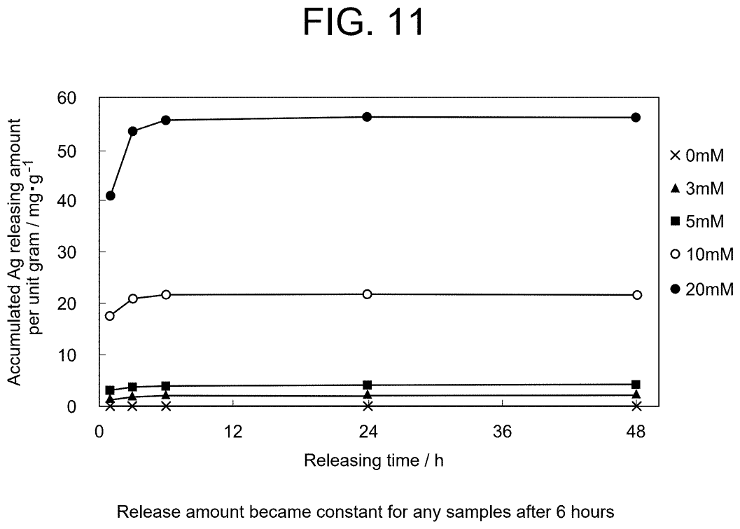

FIG. 11 shows the results of measurements of silver ion elution over time for samples of bone-regenerating materials (ReBOSSIS®) loaded with different amounts of silver (silver nitrate aqueous solution concentrations: 0, 3, 5, 10, 20 mM) according to FIG. 8. The eluted amount was almost constant when any of the samples differing in the amount of silver loaded was immersed in HEPES buffer for more than 6 hours. Since the elution amount of silver ions and the antimicrobial property are deeply dependent on the silver ion concentration, this result shows that the antimicrobial property can be easily controlled by the concentration of the silver nitrate aqueous solution used, and it is considered to be effective for the prevention of infection in the early stage after the operation.

By selecting the concentration of the silver nitrate aqueous solution within the range examined in this study, the amount of silver loaded per 1 g of bone regeneration material can be controlled from 0 to 50 mg, and it has been revealed that the amount of silver ions loaded increases as the concentration of the immersed silver nitrate aqueous solution increases.

### <Antimicrobial Assessment>

IP6 surface-modified bone regeneration materials (ReBOSSIS) were immersed in an aqueous silver nitrate solution (concentrations 0, 1.25, 2.5, 5.0 mM) to support silver to produce samples IP6_ReBO(0), IP6_ReBO(1.25), IP6_ReB)(2.5), and IP6_ReBO(5.0), and the antimicrobial properties of each sample were evaluated by two methods: the I. Shake method and the II. Inhibition Circle method.

Antimicrobial Assessment by I. Shake Method

Medium: LB medium (1×, 1/10×)

Samples: LB medium, IP6 surface-modified to silver-loaded

IP6_ReBO(0)IP6_ReBO(1.25); IP6_ReBO(2.5); IP6_ReBO(5.0)

Escherichia coli (E. coli)

Bacterial count: 1×10 5 cells/tube

Preparation of Microbial Solution

1) Control

9 ml of LB culture medium is placed into the 50 ml centrifuge pipe, and a further 1 ml of the fungal solution prepared in 1×105 CFU/ml is added to produce the suspension.

2) Sample

Prepare 9 ml of extract, add 1 ml of bacterial suspension prepared at 1×10 5 CFU/ml, and prepare a suspension.

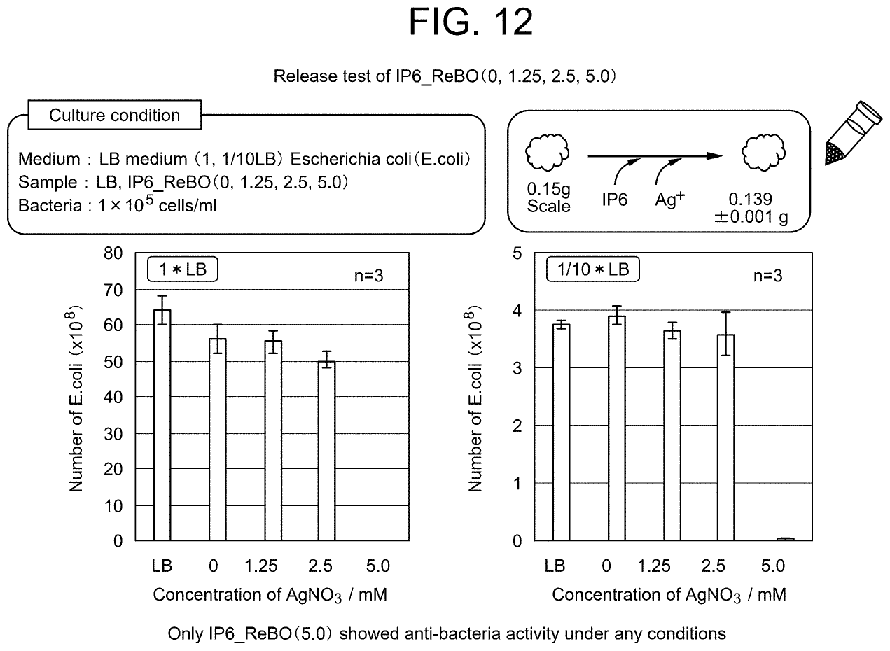

Set a 50 ml centrifuge tube filled with a microbial solution in a shaker at 37° C. and start culture. After 24 hours, the bacterial suspension was collected from a 50 ml centrifuge tube and the turbidity was measured using a spectrophotometer. FIG. 12 shows the results of evaluating the antimicrobial properties based on the relationship between the absorbance and the number of bacteria. As shown in FIG. 12, only the sample IP6_ReBO (5.0) exhibited antimicrobial properties under all conditions. From these results, it was found that AgNO3 concentration was 5.0 mM or more, regardless of the concentration of the LB medium, and the LB medium exhibited antimicrobial properties.

### II. Antimicrobial Evaluation by the Inhibition Circle Method

IP6_ReBO(0), IP6_ReBO(1.25) and IP6_ReBO(2.5) of materials for regeneration of fluffy bones (ReBOSSIS®),

0.15 g of a sample of IP6_ReBO (5.0) was filled into a mold former and pressure-molded to prepare a disc sample piece. After sterilizing the sample pieces, each of the samples was evaluated for antimicrobial properties by using the inhibition circle method.

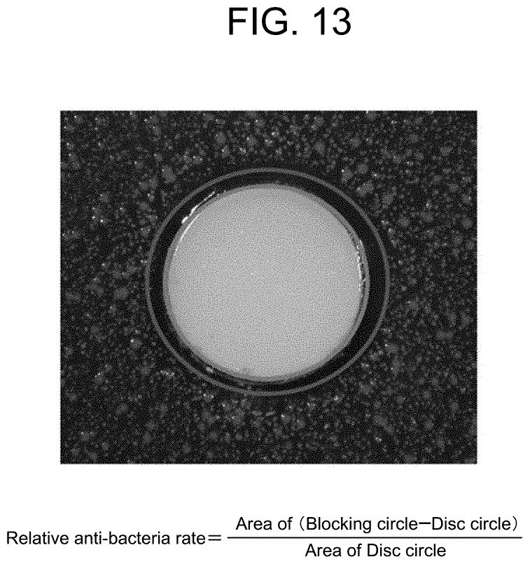

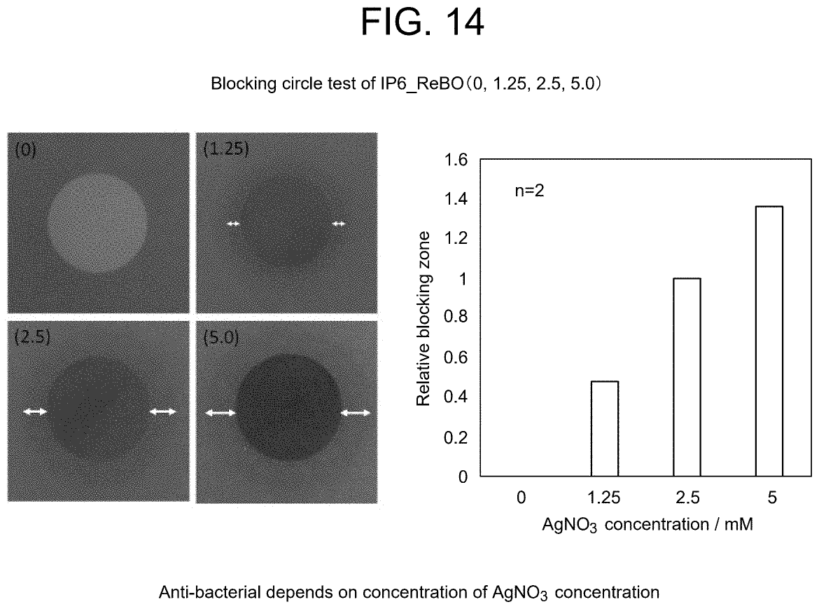

Specifically, the aforementioned disc sample pieces were placed on LB-agar medium, to which top agar containing E. coli prepared to be 1×106 CFU/plate was overlaid. Antimicrobial properties were evaluated by observing the formation of inhibition circles after 48 hours of incubation at 37° C. and comparing the relative antimicrobial rates (FIG. 13). When the silver ion was not immobilized, that is, when E. coli was cultured on IP6_Rebo (0), the bacteria grew around disc, and the formation of the inhibition circle was not observed. On the other hand, in all of IP6_Rebo (1.25, 2.5, 5.0), colony formation of E. coli was not observed in the periphery of the sample piece, and formation of a blocking circle was observed (FIG. 14).

In addition, samples IP6_ReBO(5), IP6_ReBO(10), and IP6_ReBO(20) were prepared by immersing in an aqueous silver nitrate solution (concentration: 5.0, 10, 20 mM) to support silver, and the same experiment was conducted, and it was found that although the areas of the blocking bands of samples IP6_ReBO(10) and IP6_ReBO(20) were about the same, the areas were larger than those of IP6_ReBO(5). This result suggests that although the silver ion to be immobilized increases depending on the concentration of the silver nitrate aqueous solution, since the amount of silver ion immobilized reaches the amount of silver ion necessary for the antimicrobial action at a concentration of 10 mM or more of the silver nitrate aqueous solution, even if the silver ion is immobilized further, a large change does not occur in the antimicrobial level.

### <Cytotoxicity Assessment>

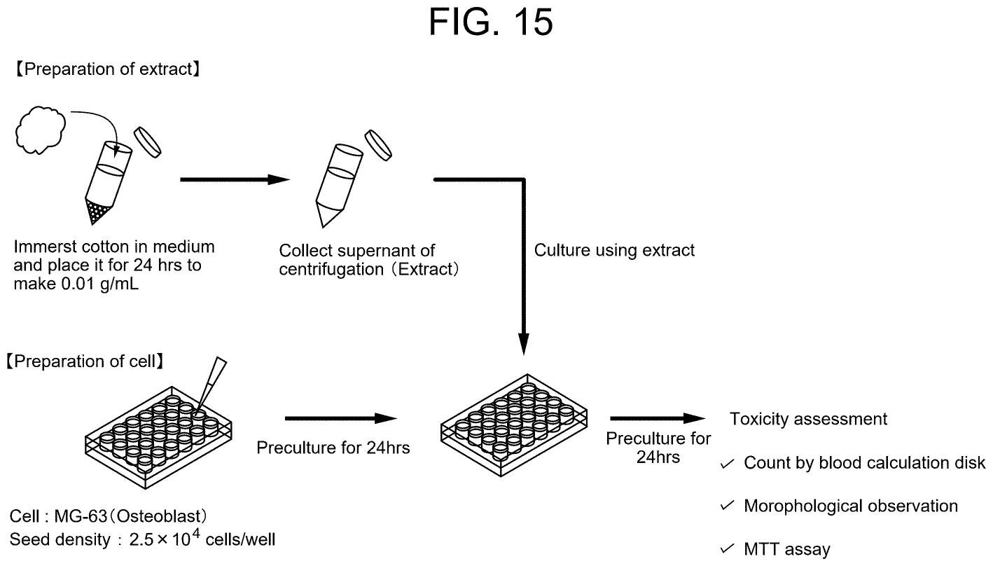

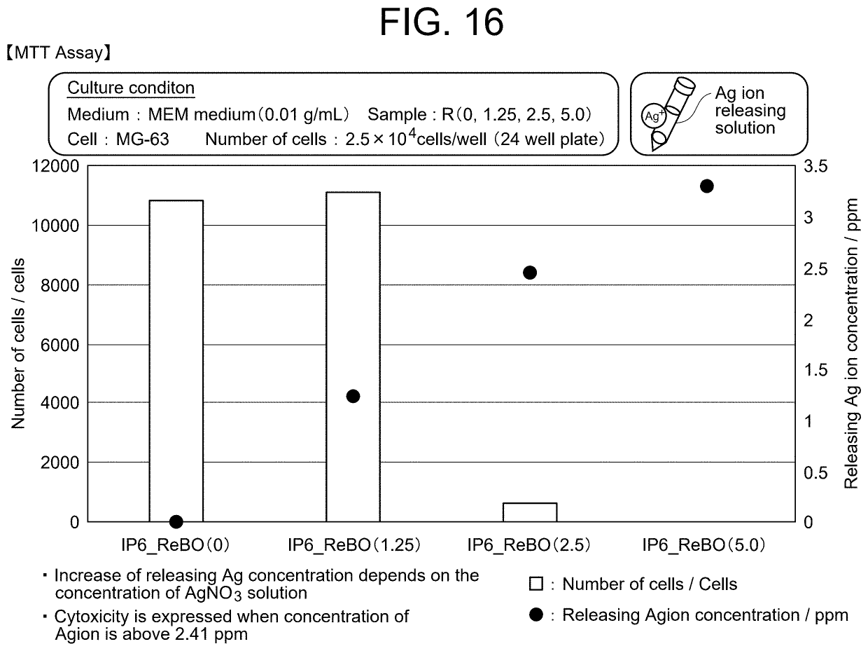

FIG. 15 shows the experimental method of the cytotoxicity test. The samples IP6_ReBO(0), IP6_ReBO(1.25), IP6_ReBO(2.5), and IP6_ReBO(5.0) are immersed in the medium for 24 hours so as to be 0.01 g/ml, respectively, and only the supernatant (extract) is collected by centrifugation. FIG. 16 is a graph of the number of cells converted from absorbance by MTT-assay after immersion of the recovered extract on osteoblasts previously prepared on Well.

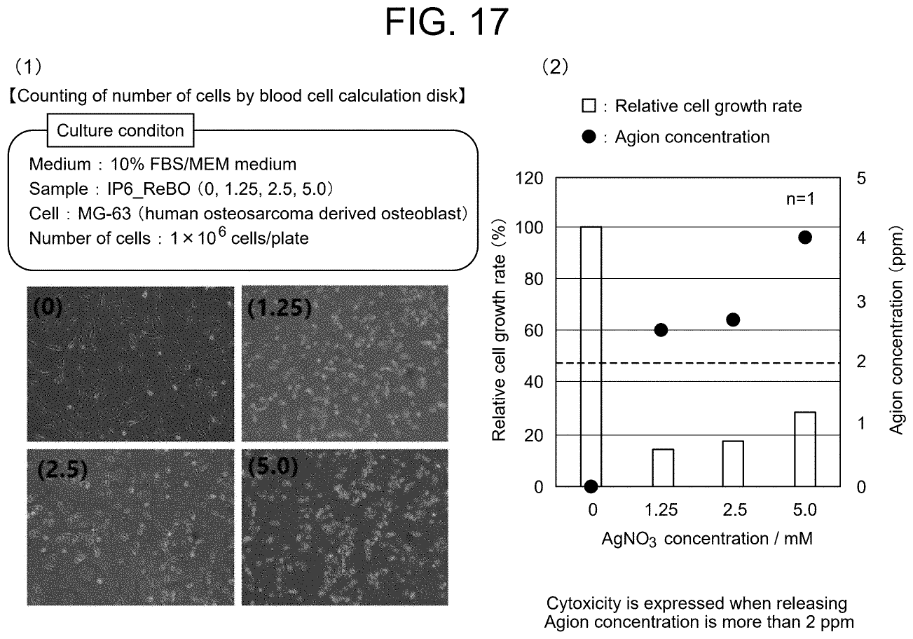

FIG. 17 is a result of directly seeding the IP6_ReBO(X) group with osteoblasts, and counting the number of the cells by the hemocytometer plate. According to FIG. 17, it is considered that cytotoxicity occurs at an eluted silver ion concentration of 2.41 ppm or more. Incidentally, this corresponds to about 2.5 mM in the immersed silver nitrate aqueous solution.

### Animal Experiment 1

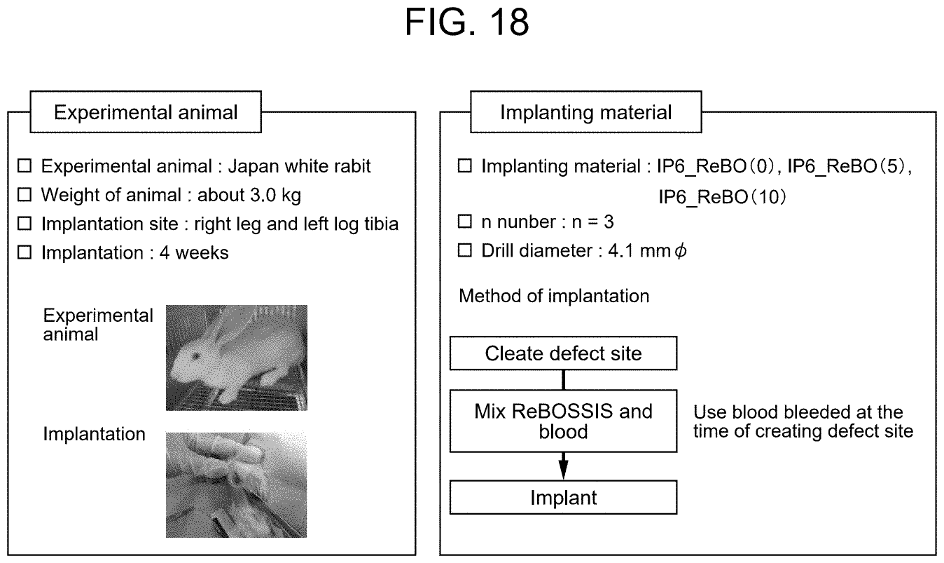

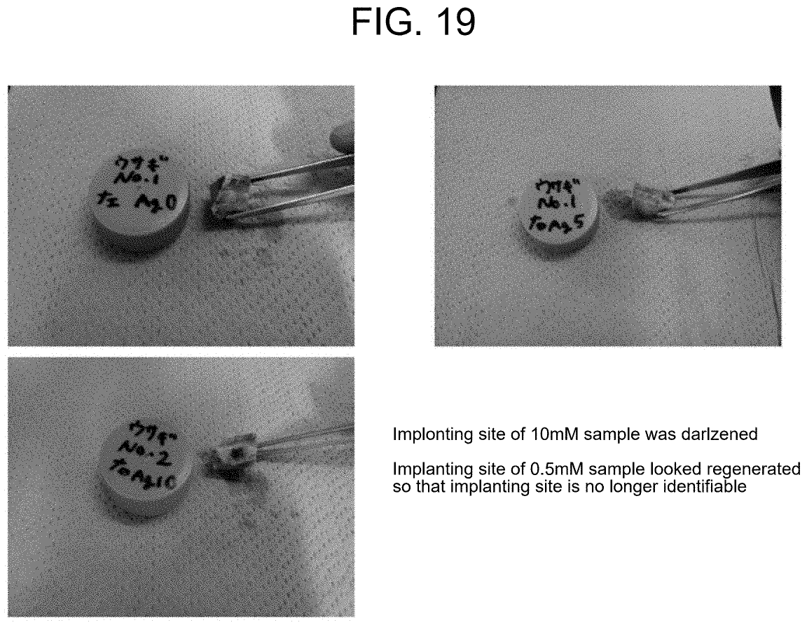

Animal experiments were performed using samples of silver-loaded bone regeneration materials (ReBOSSIS) using IP6 to evaluate biocompatibility. 4.1 mm diameter defect was made using drills in the right and left paw tibiae of male Japan white rabbits weighing about 3 kilograms and samples (silver-loaded cotton-shaped bone regeneration materials at 0, 5, and 10 mM aqueous silver nitrate concentrations, respectively) were implanted. At the time of implantation, the blood and the sample material which came out at the time of making the defect were mixed and then implanted. The implantation period was 4 weeks, and then number of each was 3 (FIG. 18). Tibia was removed from the rabbits after the implantation period, and each evaluation analysis was performed. FIG. 19 shows a photograph when the embedded sample is taken out. The 10 mM silver-loaded sample was darkened in what appeared to be an implant site. This color is believed to originate from the supported silver. The 0.5 mM silver-loaded samples were found to have been repaired such that implants portion cannot be identified.

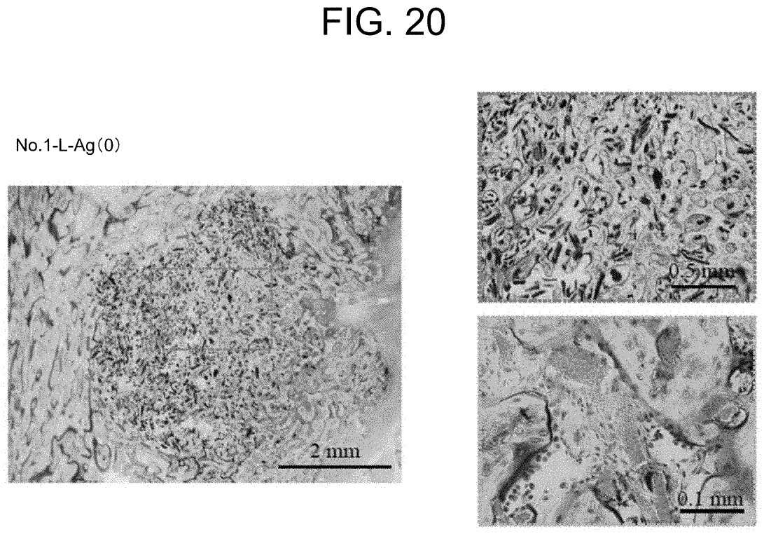

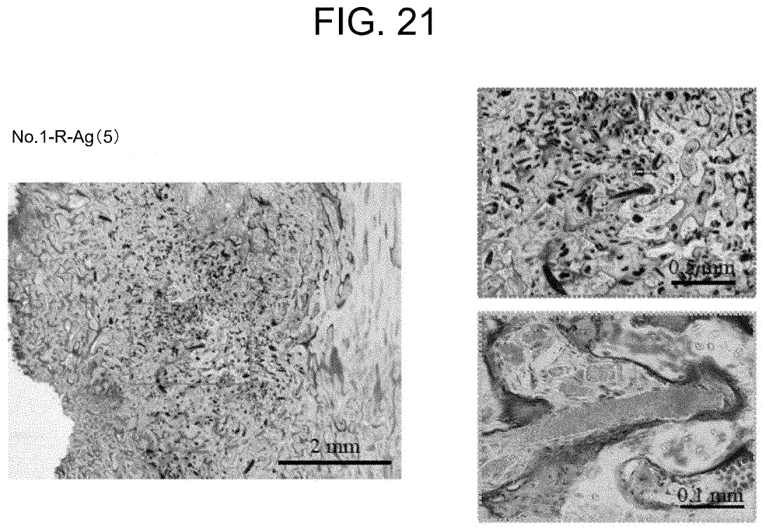

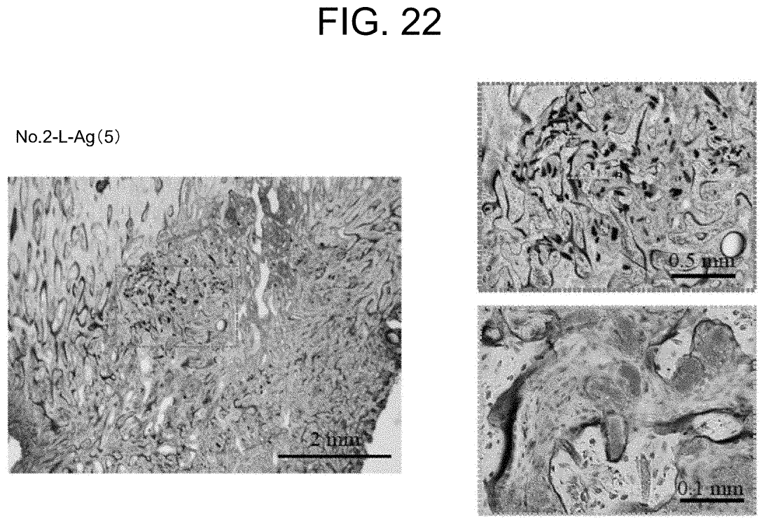

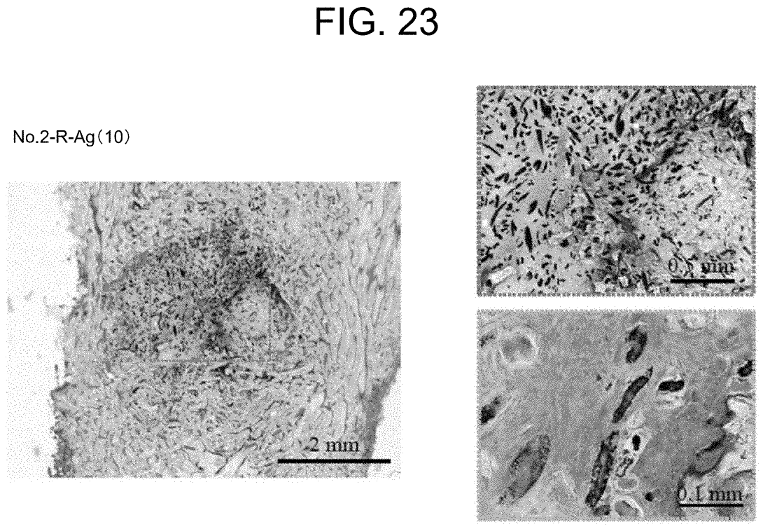

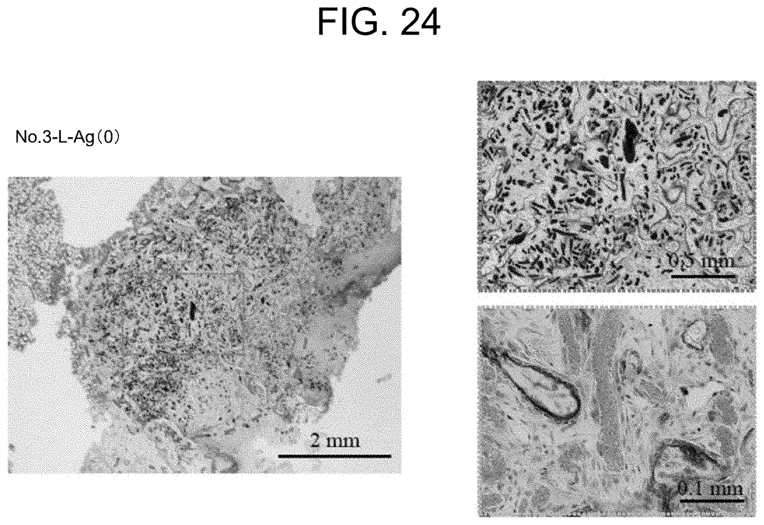

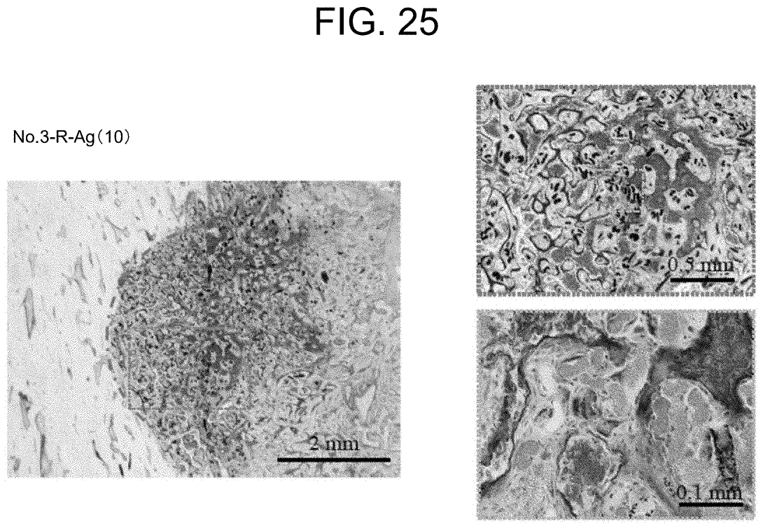

FIGS. 20-25 show histological evaluation by pathological sections. The photograph (bright-field) is shown for each of the following: the left foot Ag(0) carrying of the first individual; the right foot Ag(5) carrying of the first individual; the left foot Ag(5) carrying of the second individual; the right foot Ag(10) carrying of the second individual; the left foot Ag(0) carrying of the third individual; the right foot Ag(0) carrying of the third individual; and the sample implantation section of the right foot Ag(10) carrying the third individual. The sample looked like a black shadow, but good penetration of the new bone in the defect was observed regardless of the loading of silver.

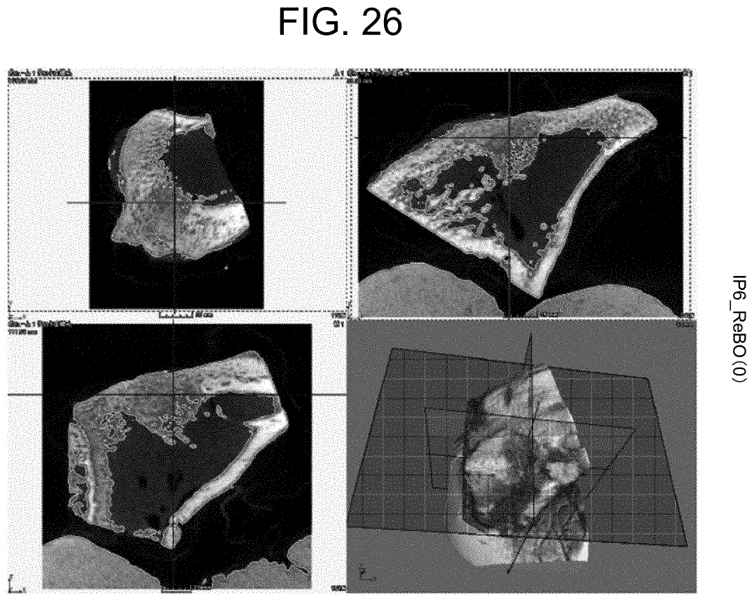

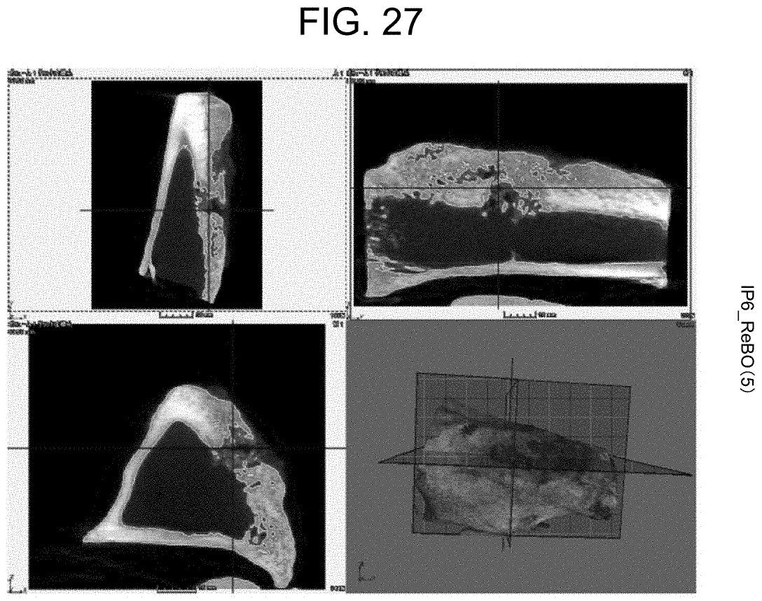

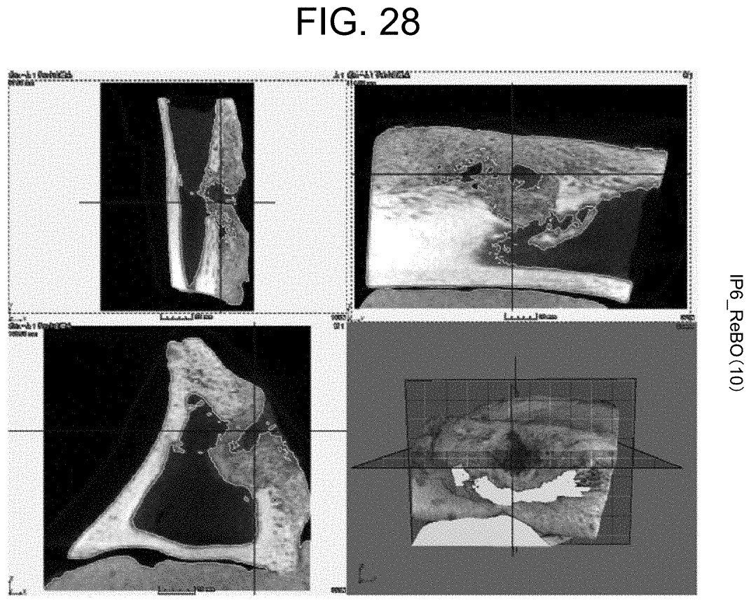

FIGS. 26-28 show the results of an animal experiment in which 0, 5, and 10 mM silver-bearing samples of the silver-bearing bone regeneration material of the present invention were implanted into rabbits as μCT scans. It was confirmed that there was no deleterious effect by silver at all silver loading concentrations, and it was found that the biocompatibility was high.

### Animal Experiment 2

Antimicrobial tests were performed in in vivo environments using “models of mouse superficial gluteus infection”. Antimicrobial cotton-shaped artificial aggregate (Ag+ion concentration: 0, 1, 5 mM anti-microbe processing) was embedded in shallow gluteal muscles of mouse organisms with light-emitting stapler (MSSA)1×10 CFU 2 μl (each n=5). The luminescence MSSA in the mice on days 1 and 3 after implantation was measured by light imaging (IVIS) and bacterial growth changes in the mice were observed.

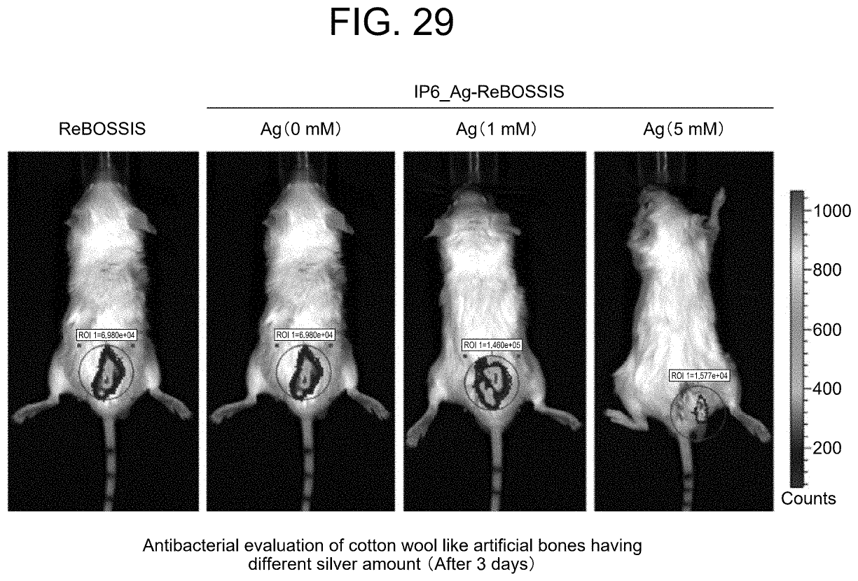

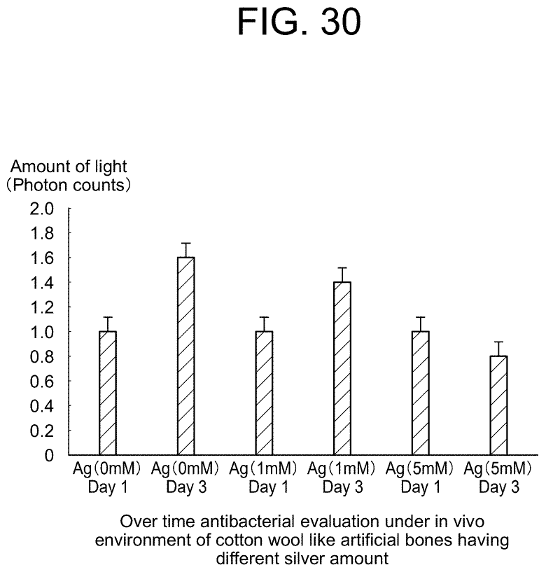

Observation results of bacterial growth changes in mice in animal experiment 2 are shown in FIGS. 29 and 30. In FIGS. 29 and 30, Ag (0, 1, 5 mM) indicates a bone regeneration material in which the fiber of the bone regeneration material in the form shown in FIG. 8 is immersed in a phytic acid (IP6) solution and washed, followed by immersion in a silver nitrate solution having concentrations of 0, 1, and 5 mM and then washing with pure water, so that silver is loaded on the fiber of the bone regeneration material in the amounts in the respective cases.

FIG. 30 shows result of numbering the light from bacteria described above. 1.6-fold PI (Photon Intensity: bacterial load) was observed from day 1 to day 3 at Ag+ion concentrations of 0 mM (control). On the other hand, bacterial growth was significantly suppressed at 1.34-fold at 1 mM and 0.78-fold at 5 mM (FIG. 29: Photograph (day 3) and FIG. 30 (day 1, day 3)). Antimicrobial tests in In vivo environments revealed that cotton-shaped artificial bones immobilized with Ag+ions with IP6 also showed antimicrobial properties in vivo.

Although the preferred embodiments for carrying out the present invention have been described above, the present invention is not limited thereto, and various modifications can be made within the scope of Technology idea of the present invention.

### Explanation of Codes

1. Biodegradable fiber

2. Calcium compound particle

3. Inositol phosphate

4. Silver ion

5. Matrix resin

1. A method for producing a bone-regeneration material having antimicrobial properties comprising biodegradable fibers, the method comprising the steps of:
immersing the biodegradable fibers in an inositol phosphate solution, wherein outer diameter of the biodegradable fiber is 10 to 100 the biodegradable fiber comprises at least 30 wt % of a biodegradable resin and 40 wt % or more of calcium compound particles, and a portion of the calcium compound particles is exposed on a surface of the biodegradable fiber, and
immersing the biodegradable fibers in a solution containing silver ions.

2. The method for producing a bone regeneration material having antimicrobial properties according to claim 1, wherein the biodegradable resin is a PLLA resin.

3. The method for producing a bone regeneration material having antimicrobial properties according to claim 1, wherein the biodegradable resin is a PLGA resin.

4. The method for producing a bone regeneration material having antimicrobial properties according to claim 1, wherein the calcium compound particles are β-phase tricalcium phosphate particles having outer diameter of 1 to 4 μm.

5. The method for producing a material for bone regeneration having antimicrobial properties according to claim 1, wherein the calcium compound particles comprises calcium carbonate or calcium phosphate.

6. The method for producing a bone regeneration material having antimicrobial properties according to claim 1, wherein the bone regeneration material containing the biodegradable fibers is formed in a cotton like shape.

7. A bone regeneration material having antimicrobial properties comprising biodegradable fibers, the biodegradable fibers having outer diameter of 10 to 100 μm, containing at least 30 wt % of a biodegradable resin and at least 40 wt % of calcium compound particles, a portion of the calcium compound particles being exposed on a surface of the biodegradable fibers, and silver ions are bound to calcium ions of the calcium compound particles that are exposed on the surface of the biodegradable fibers via inositol phosphate, whereby silver is substantially uniformly distributed and immobilized to the surface of the biodegradable fibers.

8. The bone regeneration material having antimicrobial properties according to claim 7, wherein the biodegradable resin is a PLLA resin.

9. The bone regeneration material having antimicrobial properties according to claim 7, wherein the biodegradable resin is a PLGA resin.

10. The material for bone regeneration having antimicrobial properties according to claim 7, wherein the calcium compound particles comprise calcium carbonate or calcium phosphate.

11. The material for bone regeneration having antimicrobial properties according to claim 7, wherein the calcium compound particles are β-phase tricalcium phosphate particles having an outer diameter of 1 to 4 μm.

12. The bone regeneration material having antimicrobial properties according to claim 7, wherein the bone regeneration material comprising the biodegradable fibers is formed in a cotton like shape.

13. The material for bone regeneration having antimicrobial properties according to claim 8, wherein the calcium compound particles comprise calcium carbonate or calcium phosphate.

14. The material for bone regeneration having antimicrobial properties according to claim 9, wherein the calcium compound particles comprise calcium carbonate or calcium phosphate.

15. The material for bone regeneration having antimicrobial properties according to claim 8, wherein the calcium compound particles are β-phase tricalcium phosphate particles having an outer diameter of 1 to 4 μm.

16. The material for bone regeneration having antimicrobial properties according to claim 9, wherein the calcium compound particles are β-phase tricalcium phosphate particles having an outer diameter of 1 to 4 μm.

17. The material for bone regeneration having antimicrobial properties according to claim 10, wherein the calcium compound particles are β-phase tricalcium phosphate particles having an outer diameter of 1 to 4 μm.

18. The bone regeneration material having antimicrobial properties according to claim 8, wherein the bone regeneration material comprising the biodegradable fibers is formed in a cotton like shape.

19. The bone regeneration material having antimicrobial properties according to claim 9, wherein the bone regeneration material comprising the biodegradable fibers is formed in a cotton like shape.

20. The bone regeneration material having antimicrobial properties according to claim 10, wherein the bone regeneration material comprising the biodegradable fibers is formed in a cotton like shape.
